(19) United States
(12) Patent Application Publication
(10) Pub. No.: US 2024/0408000 A1
(43) Pub. Date: Dec. 12, 2024

(54) COSMETIC COMPOSITION INCLUDING MICRONEEDLES IMPREGNATED WITH CICA EXTRACT AS ACTIVE INGREDIENT AND METHOD FOR MANUFACTURING THE SAME

(71) Applicant: VT Co., Ltd., Paju-si (KR)

(72) Inventor: Cheol JEONG, Seoul (KR)

(73) Assignee: VT Co., Ltd., Paju-si (KR)

(21) Appl. No.: 18/458,104

(22) Filed: Aug. 29, 2023

(30) Foreign Application Priority Data
Jun. 9, 2023 (KR) 10-2023-0073980

(51) Int. Cl. A61K 8/9789; A61K 8/02; A61Q 19/00

(52) U.S. Cl. CPC A61K 8/9789; A61K 8/0204; A61Q 19/00; A61K 2800/28; A61K 2800/805

(57) ABSTRACT

Disclosed is a cosmetic composition including microneedles impregnated with a cica extract as an active ingredient, and a method for manufacturing the same. The composition provides elasticity and regenerative power to the skin, and has an excellent effect of improving skin turnover, skin absorption amount, absorption depth, absorption rate, and smoothing skin texture, that is, skin roughness whereby the cosmetic composition may be usefully used.

### CROSS-REFERENCE TO RELATED APPLICATIONS

A claim for priority under 35 U.S.C. § 119 is made to Korean Patent Application No. 10-2023-0073980 filed Jun. 9, 2023 in the Korean Intellectual Property Office, the entire contents of which are hereby incorporated by reference.

### BACKGROUND

Embodiments of the inventive concept described herein relate to a cosmetic composition including microneedles impregnated with a cica extract as an active ingredient, and a method for manufacturing the same.

Skin is the largest organ that protects various organs of the body from external stimuli and plays various physiological roles. However, with age, a physiological function of skin and its function as a barrier deteriorate due to various internal and external factors, and a skin damage easily occurs even by small external stimuli. Accordingly, it is necessary to develop cosmetics for reducing skin damage caused by external stimuli.

As the technology development has extended the average lifespan of humans and increased interest in skin and appearance, a market size for functional cosmetics is steadily growing. The skin is a primary defense organ of humans against various harmful substances and is an important immune organ. Risk factors for the skin are gradually increasing, and a rate of formation and exfoliation of the stratum corneum slows down due to changes in eating habits, and moisturizing factors and a lipid content of the stratum corneum decreases due to the deterioration of the function of keratinocytes whereby the number of people with skin that cannot exhibit normal skin functions increases.

This breakdown of the skin barrier function causes not only cosmetic problems such as skin blackening, psoriasis, and excessive exfoliation of keratin, but also problems of skin diseases such as xeroderma and atopic dermatitis. However, most of these problems can be prevented or alleviated by constantly maintaining skin moisture, imparting firmness and flexibility to the stratum corneum, and adjusting a degree of contraction of surrounding muscle tissues.

Even with healthy skin, because the skin becomes dry, rough, and crumbly due to various causes such as a harsh external environment, wind, a cold weather, the sunlight, facial washing, shaving, various stresses, and natural skin aging due to age, it is very important to properly control a moisture content of the stratum corneum of the skin, a contractility of muscle cells, and a protein activity.

Recently, various types and forms of cosmetics have been used. Cosmetics have beneficial functions for the skin, such as skin cleanliness, moisturizing, UV protection, wrinkle improvement, whitening, and soothing, but they can be a main cause of contact dermatitis. Contact dermatitis (contact dermatitis) is dermatitis caused by contact with external substances, and is classified into irritant contact dermatitis caused by irritants and allergic contact dermatitis occurring in people sensitized to specific antigens. Accordingly, a safety test of cosmetics has an important significance for the purpose of reducing these side effects.

Dermatitis caused by cosmetics appears in less than 2% of users, and among them, allergic contact dermatitis accounts for 5 to 10% of side effects caused by cosmetics, and is about 0.3 to 3% of patients visiting a dermatologist. Cosmetics are manufactured by mixing various ingredients, and because they are used for a long period of time as well as in direct contact with the human body, the safety of the cosmetics itself as well as the safety of the raw materials should be secured.

Conventional cosmetics include glycerin, sorbitol, and the like, and they feel unpleasant when used due to their excessive stickiness. In addition, sodium pyrrolidone carboxylate, sodium lactate, and the like, which are known as natural additives, have strong electrolytic properties whereby it is difficult to maintain stability in the formulation. Because conventional natural-derived raw materials have many problems such as insignificant effects or stability in formulations, and side effects caused by skin irritation, it is necessary to develop a cosmetic composition that is not harmful to the human body and has excellent effects.

Under this background, the inventors finished the inventive concept after confirming that the cosmetic composition containing microneedles impregnated with a cica extract manufactured by the manufacturing method of the inventive concept as an active ingredient provides elasticity and regenerative power to the skin and confirming that the excellent effect of improving skin turnover, skin absorption amount, absorption depth, absorption speed, and smoothing skin texture, that is, skin roughness.

### SUMMARY

Embodiments of the inventive concept provides a cosmetic composition including microneedles impregnated with a cica extract as an active ingredient, and a method for manufacturing the same.

Embodiments of the inventive concept provides a method for manufacturing a cosmetic composition, including i) collecting sponge and then hydrolyzing them, ii) separating microneedles by sieving the hydrolyzed sponges, iii) drying the microneedles; and iv) agitating the dried microneedle and cica extract under a reduced pressure to impregnate the cica extract into inner tubes of the microneedles.

A cica extract of the inventive concept is also called a Centella asiatica extract, and is a perennial plant belonging to the Apiaceae family. The stem extends laterally, and has two degenerated scale-like leaves, and the leaves have a diameter of 2 to 5 cm. It blooms reddish-purple flowers and produces flat, circular fruits. It is known to have effects such as wound healing, antioxidant, promotion of blood circulation, and improvement of vascular health.

The microneedle of the inventive concept may be a spicule, and the spicule is a fine structure constituting the skeleton of a sponge living in freshwater, and is a natural composite material composed of calcium and silicate in the form of fine needles collected from seaweed or corals.

The microneedle of the inventive concept may have a length of 200 to 300 μm and a diameter of 10 to 20 μm, in detail, a length of 230 to 280 μm and a diameter of 12 to 18 μm, in more detail, a length of 10 to 20 μm. 250 μm, and may be 15 μm in diameter. When the size of the microneedle is less than the above range, a large amount of penetration into the skin may cause inflammation, and when the size exceeds the above range, the effect of improving skin regeneration and self-regeneration is insignificant.

The pH of the microneedle of the inventive concept may be 7 to 8, in detail 7 to 7.5, and in more detail 7, but is not limited thereto.

The microneedles of the inventive concept may be derived from sponges, and the spontes may be Spongilla lacustris, Spongilla fragilis leidy or Ephydatia fluviatilis.

The content ratio of a cica extract and microneedles in the inventive concept may be 0.5 to 2:98 to 99.5 parts by weight, in detail 0.5 to 1.5:98.5 to 99.5 parts by weight, and in more detail 1:99. If the content range of the cica extract and the microneedles is outside the above range, the cica extract may not be sufficiently contained in the microneedles.

The cosmetic composition of the inventive concept may have an effect of improving skin turnover. In a specific embodiment of the inventive concept, the general skin turnover cycle is 4 weeks or more, but when the cosmetic composition of the inventive concept is used, it was confirmed that it is shortened to 2 weeks whereby skin turnover was promoted.

The cosmetic composition of the inventive concept may have an effect of improving skin regeneration. In a specific embodiment of the inventive concept, as a result of measuring the improvement in skin regeneration after use of the cosmetic composition of the inventive concept, it was confirmed that the measured value of the amount of erythema in the cosmetic composition use part decreased while showing a statistically significant difference 24 hours after UV irradiation as compared with the nonuse part.

The cosmetic composition of the inventive concept may have an effect of increasing skin absorption amount, absorption depth, and absorption rate. In a specific embodiment of the inventive concept, as a result of measuring skin absorption after use of the cosmetic composition of the inventive concept, it was confirmed that the measured value of skin absorption amount, absorption depth and absorption rate increased statistically significantly after use of the cosmetic composition as compared with before use.

The cosmetic composition of the inventive concept may have an effect of promoting improvement of the skin roughness. In a specific embodiment of the inventive concept, as a result of measuring skin roughness, that is, skin texture, after use of the cosmetic composition of the inventive concept, it was confirmed that the skin roughness analysis value decreased statistically significantly immediately after use as compared with before use of the cosmetic composition whereby the skin texture was improved.

The cosmetic composition of the inventive concept may be manufactured of formulations selected from a group consisting of solutions, external ointments, creams, foams, nourishing lotions, softening lotions, perfumes, packs, softening waters, emulsions, makeup bases, essences, soaps, liquid cleansers, bath additives, sunscreen creams, sun oils, suspensions, emulsions, pastes, gels, lotions, powders, surfactant-containing cleansers, oils, powder foundations, emulsion foundations, wax foundations, patches and sprays, but is not limited thereto.

The cosmetic composition may further include one or more cosmetically acceptable carriers formulated in general skin cosmetics, and as typical ingredients, for example, oil, water, surfactants, moisturizers, lower alcohols, thickeners, and chelating agents, Colorants, preservatives, flavorings, and the like may be appropriately blended, but are not limited thereto.

Cosmetically acceptable carriers included in the cosmetic composition of the inventive concept vary according to the formulation of the cosmetic composition.

When the formulation of the inventive concept is ointment, paste, cream or gel, animal oil, vegetable oil, wax, paraffin, starch, tracanthate, cellulose derivative, polyethylene glycol, silicone, bentonite, silica, talc, zinc oxide, and the like may be used as a carrier component, but is not limited thereto. These may be used alone or in combination of two or more.

When the formulation of the inventive concept is a powder or spray, lactose, talc, silica, aluminum hydroxide, calcium silicate, polyamide powder, and the like may be used as a carrier component, and in particular, in the case of a spray, a propellant, such as chlorofluorohydrocarbon, propane/butane or dimethyl ether, may be further included, but is not limited thereto. These may be used alone or in combination of two or more.

When the formulation of the inventive concept is a solution or emulsion, a solvent, solubilizing agent or emulsifying agent may be used as a carrier component, for example, water, glycerin, ethanol, isopropanol, ethyl carbonate, ethyl acetate, benzyl alcohol, benzyl benzoate, propylene glycol, 1,3-butyl glycol oil, and the like may be used, and in particular, cottonseed oil, peanut oil, corn germ oil, olive oil, castor oil and sesame oil, glycerol aliphatic ester, polyethylene glycol or sorbitan fatty acid esters of may be used, but is not limited thereto. These may be used alone or in combination of two or more.

When the formulation of the inventive concept is a suspension, a liquid diluent such as water, glycerin, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar or tracanth, and the like may be used as carrier components, and are not limited thereto. These may be used alone or in combination of two or more.

When the formulation of the inventive concept is a soap, alkali metal salts of fatty acids, fatty acid hemiester salts, fatty acid protein hydrolyzates, isethionates, lanolin derivatives, fatty alcohols, vegetable oils, glycerol, sugars, and the like can be used as carrier components, but are not limited thereto. These may be used alone or in combination of two or more.

Another aspect of the inventive concept provides a method for manufacturing a cosmetic composition, including i) collecting sponge and then hydrolyzing them, ii) separating microneedles by sieving the hydrolyzed sponges, iii) drying the microneedles; and iv) agitating the dried microneedle and cica extract under a reduced pressure to impregnate the cica extract into inner tubes of the microneedles.

In operation i), the hydrolysis may be performed for 30 to 50 hours, but is not limited thereto.

In operation ii), the sieving may be performed with a 50 to 200 mesh, and in detail, may be performed with a 50 to 150 mesh, and in more detail, may be performed with a 100 mesh, but is not limited thereto.

In operation iii), the drying may be performed at 70 to 100° C. for 20 to 30 hours, and in more detail, at 70 to 90° C. for 24 hours.

In operation iv), the reduced pressure agitation may be performed at −0.1 to 0.1 MPa for 2 to 5 hours, in detail, at −0.07 to −0.05 MPa for 2 to 4 hours, and in more detail, at −0.06 MPa for 3 hours. The cica extract can be sufficiently impregnated into the inner tubes of the microneedles within the pressure and time range of the reduced-pressure agitation.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a view illustrating changes in skin color in a result of measurement of a skin turnover expediting effect after a cosmetic composition of the inventive concept is used;

FIG. 2 is a view illustrating skin exfoliation rates in a result of measurement of a skin turnover expediting effect after a cosmetic composition of the inventive concept is used;

FIG. 3 is a view illustrating skin erythema amount measurement values in a result of measurement of a skin regeneration improving effect after a cosmetic composition of the inventive concept is used;

FIG. 4 is a view illustrating a result obtained by measuring skin absorption amounts after a cosmetic composition of the inventive concept is used; and

FIG. 5 is a view illustrating a result obtained by measuring roughness of skin, that is, textures of skin after a cosmetic composition of the inventive concept is used.

### DETAILED DESCRIPTION

The advantages and features of the inventive concept, and a method for achieving them will become apparent from the following description of the following embodiments given in conjunction with the accompanying drawings. However, the inventive concept is not limited by the embodiments disclosed herein but will be realized in various different forms, and the embodiments are provided only to make the disclosure of the inventive concept complete and fully inform the scope of the inventive concept to an ordinary person in the art, to which the inventive concept pertains, and the inventive concept will be defined by the scope of the claims.

The terms used herein are provided to describe the embodiments but not to limit the inventive concept. In the specification, the singular forms include plural forms unless particularly mentioned. The terms “comprises” and/or “comprising” used herein does not exclude presence or addition of one or more other components, in addition to the aforementioned components. Throughout the specification, the same reference numerals denote the same components, and “and/or” includes the respective components and all combinations of the components. Although “first”, “second” and the like are used to describe various components, the components are not limited by the terms. The terms are used simply to distinguish one component from other components. Accordingly, it is apparent that a first component mentioned in the following may be a second component without departing from the spirit of the inventive concept.

Unless otherwise defined, all terms (including technical and scientific terms) used herein have the same meaning as commonly understood by those skilled in the art to which the inventive concept pertains. It will be further understood that terms, such as those defined in commonly used dictionaries, should not be interpreted in an idealized or overly formal sense unless expressly so defined herein.

Hereinafter, the contents of the inventive concept will be described in more detail through the following embodiments and experimental examples. However, the scope of the inventive concept is not limited only to the following embodiments and experimental examples, and includes modifications of equivalent technical spirits.

### First Embodiment Manufacturing of Cosmetic Composition

### 1-1. Collection of Raw Material

Freshwater Sponge from Ukraine, which live in fresh water, was used as a raw material. The raw material was directly imported and used for purification.

The Freshwater Sponge is also called hydrolyzed sponge or spicule, and the spicule is a very small needle in the form of a needle extracted from freshwater sponge, and penetrates deeply into skin to stimulate the dermis and activate skin microcirculation to help converge skin pores.

### 1-2. Hydrolysis of Raw Material

The raw material was agitated for 30 minutes. When the temperature gradually rises to 30 to 40° C., hydrolysis is performed while agitating it for 48 hours.

### 1-3. Primary Sieving and Washing

The raw material hydrolyzed in operation 1-2 was sieved with a 100 mesh such that the spicule was separated, and the spicule was washed 5 times.

### 1-4. Drying and Secondary Sieving

It was dried for 24 hours at 70 to 90° C. Thereafter, it was sieved by a 200 mesh such that foreign substances were removed.

### 1-5. Coating of Centella Extract

Spicules and centella extract dried in a vacuum agitator were introduced at a ratio of 1:1. The temperature of the vacuum agitator was set to 80° C., and a vacuum pump was started to heat an interior of a tank while the pressure was reduced to −0.06 MPa, and it was agitated for 3 hours and was dried, and at the same time, the centella extract was impregnated into an inner tube of the spicule. The cosmetic composition of the inventive concept includes centella extract and spicule in a content ratio of 1:99 parts by weight.

### 1-6. Packing and Sterilization

After primary packing with a zipper bag, secondary packing was performed with an aluminum pack. After sterilization through a gamma treatment, a cosmetic composition was finally manufactured.

### Experimental Example 1. Improvement of Turnover of Skin

### 1-1. Test Subject Information

The information on the test subjects who participated in the test is as in Table 1 as follows.

TABLE 1

| | |
|---|---|
| Starting text subjects | 22 persons |
| Dropouts | 2 persons |
| Reasons of dropout | No. 13, V3 dropout - tracking failed |
| | No. 20, V4 dropout - tracking failed |
| Ending test subjects | 20 persons |
| Sex | Female - 21 persons, Male - one person |
| Average age | 48.591 ± 8.273 years old |

### 1-2. Test Method

For a total of 22 test subjects, a designated test part was divided (2×2 cm) into used and unused parts of the manufactured cosmetic composition. Thereafter, the skin was measured and photographed before dyeing, and 2.0 mg/cm2 of 8% dihydroxyacetone (DHA) emulsion was applied to the parts to dye the skin keratin. For two weeks, the composition was used only on the used part of the cosmetic composition of the inventive concept, and the skin was measured and photographed before and after dyeing, one day, seven days, and fourteen days before and after use of the product.

### 1-3. Evaluation Method

Improvement of a turnover of skin was measured and evaluated through measurement of a skin color and skin UV photography. To measure the skin color, Chromameter CR 400 was used to measure the skin color (L value) of each part before and after dyeing, one day, seven days, and fourteen days after using the product. The skin color was measured three times for each part, and the average value was substituted into the following Equation to calculate a keratin exfoliation rate (%).

$$Keratin exfoliation rate ( \% ) = \frac{\begin{matrix}Measured value of skin color ( value L ) \\ n days after use of product - \\ measured value of skin color ( value L ) after dyeing\end{matrix}}{\begin{matrix}Measured value of skin color ( value L ) before dyeing - \\ measured value of skin color ( value L ) after dyeing\end{matrix}} \times 100$$

It means that the skin turnover expediting effect becomes better as the value of the exfoliation rate of the dyed skin after use of the product becomes higher.

Furthermore, Janus 1 Mark II was used for skin UV photography.

### 1-4. Test Result

This test was conducted on 22 male and female adults between the ages of 19 and 60, and a total of 20 test subjects, excluding 2 dropouts, used the cosmetic composition of the test product, the inventive concept, on the forearm for 2 weeks.

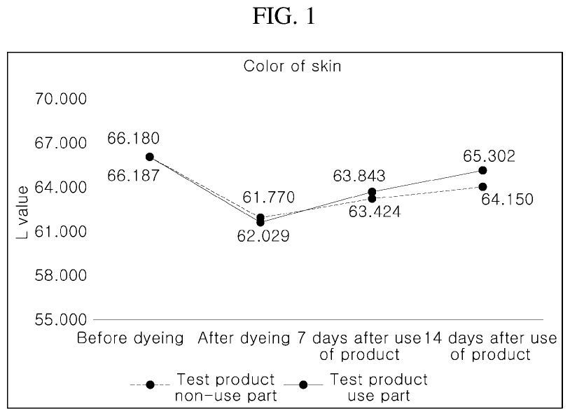

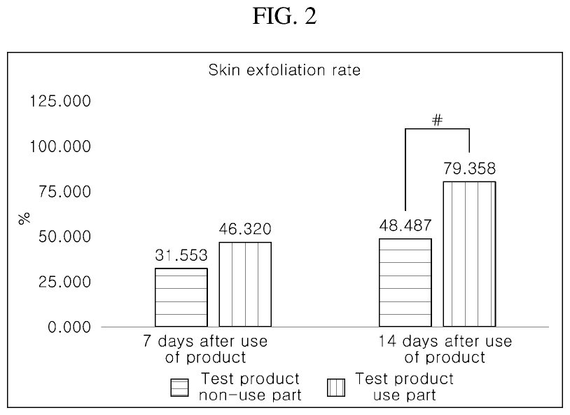

As a result of measuring the skin turnover expediting effect after use of the test product, it was confirmed that the measurement value of the keratin exfoliation rate at the part, in which the test product was used, was improved with a statistically significant difference after fourteen days of use of the product as compared with the part, in which the test product was not used (p<0.05). The skin color and the skin exfoliation rate are as illustrated in FIGS. 1 and 2.

It was confirmed that the keratin exfoliation rate value of the dyed skin increased after use of the product (Table 2).

TABLE 2
Change in skin color and keratin exfoliation rate

| | | | | Significance |
|---|---|---|---|---|
| | | | Keratin | probability |
| | Measured | Average ± standard | exfoliation | Comparison of |
| Time | part | deviation | rate | groups |
| Before | Non-use of | 66.187 ± 1.657 | — | — |
| dyeing | product | 66.180 ± 2.162 | — | |
| | Use of | | | |
| | product | | | |
| After | Non-use of | 62.029 ± 2.115 | — | — |
| dyeing | product | 61.770 ± 2.201 | — | |
| | Use of | | | |
| | product | | | |
| 7 days | Non-use of | 63.424 ± 2.482 | 31.553 | 0.163 |
| after | product | 63.843 ± 2.503 | 46.320 | |
| use of | Use of | | | |
| product | product | | | |
| 14 days | Non-use of | 64.150 ± 2.590 | 48.487 | 0.005# |
| after | product | 65.302 ± 2.605 | 79.358 | |
| use of | Use of | | | |
| product | product | | | |

Keratin exfoliation rate^{a}(%)={[Measure value (value L) of skin color after n days after use of product - measured value of skin color (value L) after dyening]/[Measured value of skin color (value L) before dyeing-measured value of skin color (value L) after dyeing]} × 100
Significance probabilityb(value p)#: p < 0.05 by Independent T-test

This indicates that the cosmetic composition of the inventive concept may help improve the skin turnover with just two weeks of use. A general skin turnover cycle is 4 weeks or more, but when the cosmetic composition of the inventive concept is used, it is shortened to 2 weeks, suggesting that the skin turnover is expedited.

### Experimental Example 2. Improvement of Regeneration of Skin

### 2-1.2-1. Test Subject Information

The information of the test subjects who participated in the test is shown in Table 1 above.

### 2-2. Test Method

For a total of 22 test subjects, a designated test part was divided (2×2 cm) into used and unused parts of the manufactured cosmetic composition. After using the cosmetic composition for 2 weeks on the regeneration test part, ultraviolet rays were irradiated, the skin was measured and photographed before ultraviolet irradiation and 24 hours after the ultraviolet irradiation.

### 2-3. Evaluation Method

To measure improvement of a regeneration of skin, a skin barrier was temporarily damaged by irradiating UV light with a wavelength of 290 to 320 nm and a light intensity of 44.22 mJ/cm² by using multiple ports. Thereafter, the skin was photographed for a general picture by using Antera 3D, and a skin erythema amount was measured by using Mexameter MX18. Skin erythema was evaluated by measuring the erythema Index (E.I) of the irradiated part before UV irradiation and 24 hours after UV irradiation. The average value measured three times was used as skin erythema evaluation data, and the unit of the measured erythema Index was an arbitrary unit (A.U). Because the measurement value and the erythema Index are proportional to each other, a lower measurement value means that the occurrence of erythema is suppressed.

### 2-4. Test Result

This test was conducted on 22 male and female adults between the ages of 19 and 60, and a total of 20 test subjects, excluding 2 dropouts, used the cosmetic composition of the test product, the inventive concept, on the forearm for 2 weeks.

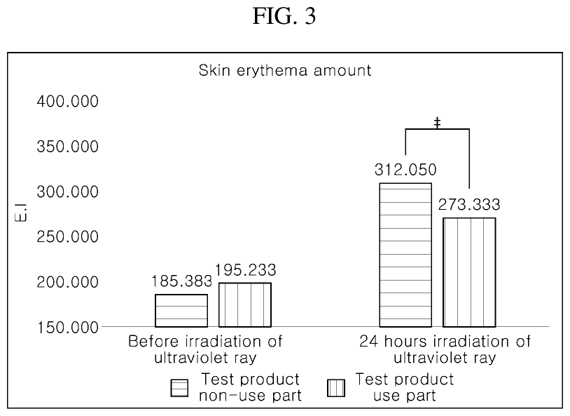

As a result of measuring improvement of a regeneration of skin after use of the test product, it was confirmed that the measurement value of the amount of skin erythema in the part, in which the test product was used, decreased with a statistically significant difference after 24 hours of UV irradiation as compared with the part, in which the test product was not used (p<0.05) (FIG. 3).

The results of measuring a skin regeneration improving effect of the cosmetic composition of the inventive concept by measuring a change in the skin erythema amount are as illustrated in Table 3 below.

TABLE 3
Change in skin erythema amount

| | | | | Significance |
|---|---|---|---|---|
| | | | | probability |
| | | Average ± standard | | (value p) |
| | Use of | deviation | Change rate^{a} | Comparison |
| Time | product | (Erythema index) | (%) | of groups |
| Before | Non-use of | 185.383 ± 243.383 | — | — |
| irradiation of | product | | | |
| ultraviolet ray | Use of | 195.233 ± 46.801 | — | |
| | product | | | |
| 24 hours after | Non-use of | 312.050 ± 77.553 | 75.146 | 0.042‡ |
| irradiation of | product | | | |
| ultraviolet ray | Use of | 273.333 ± 62.912 | 46.937 | |
| | product | | | |

$Change {rate}^{a} ( \% ) = {\sum }_{i = 1}^{n} \frac{\begin{matrix}n time after radiation of ultraviolet ray i - \\ before radiation of ultraviolet ray i\end{matrix}}{before radiation of ultraviolet ray i} * 100 / n ,$ n = the number of test subjects
Significance probability\hat{ }b (value p)
‡p <0.05 by Mann-Whitney U test

This suggests that the cosmetic composition of the inventive concept has a remarkable effect on improving a regeneration of skin with 2 weeks of use.

### Experimental Example 3. Improvement of Absorption of Skin

### 3-1. Test Subject Information

The information of the test subjects who participated in the test is as in Table 4 below.

TABLE 4

| | |
|---|---|
| Starting text subjects | 20 persons |
| Dropouts | 0 persons |
| Reasons of dropout | — |
| Ending test subjects | 20 persons |
| Sex | Female 20 persons |
| Average age | 46.500 ± 7.287 years old |

### 3-2. Measurement of Absorption Amount of Skin

### 3-2-1. Measurement Method

The skin absorption amount was measured by dividing the forearm of the test subject into a test product non-use part, an essence use part, and a test product use part, and then, by using gen2-SCA performance, a skin absorption amount of each part before use of the product, and 20 minutes after the use of the product.

The measured data was analyzed using by Skin Tools 3.0, and the value measured once was used as skin absorption evaluation data. The unit of the measured skin absorption amount is expressed by μg/cm³, and because the measurement value and the degree of skin absorption are proportional to each other, a higher the measurement value of the skin absorption amount means a greater absorption into the skin.

### 3-2-2. Measurement Result

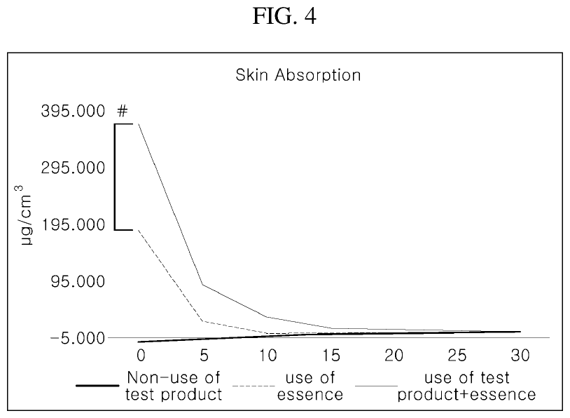

As a result of measuring a skin absorption after use of the test product and essence, it was confirmed that the measurement value of the skin absorption increased statistically significantly after use of the product as compared with before use of the product (p<0.05) (FIG. 4). Furthermore, the amount of change in the total skin absorption measurement value of the test product and essence use part showed a statistically significant difference after use of the product as compared with the essence use part (p<0.05).

### 3-3. Measurement of Skin Absorption Depth

### 3-3-1. Measurement Method

The depth of skin absorption was measured by dividing the forearm of the test subject into a test product non-use part, an essence use part, and a test product use part and then using gen2-SCA performance, the skin absorption of each area before use of the product and 20 minutes after the use of the product use was measured. The depth, at which the skin absorption amount was statistically significantly increased after use of the product as compared with before use of the product, was used as skin absorption depth evaluation data. The unit of the measurement value is expressed as μm, and a higher measurement value means that it is absorbed into the skin deeper.

### 3-3-2. Measurement Result

As a result of measuring a skin absorption depth after use of the test product and essence, it was confirmed that the skin absorption depth increased statistically to a depth of 25 to 30 μm 20 minutes after the use of the product (p<0.05).

### 3-4. Measurement of Skin Absorption Rate

### 3-4-1. Measurement Method

The skin absorption rate was confirmed by dividing the skin absorption depth for each part by the absorption time, and because the calculated value and the degree of improvement of the skin absorption rate are proportional to each other, ae higher calculated value means a faster absorption.

### 3-4-2. Measurement Result

As a result of calculating the skin absorption rate after using the test product and essence, the skin absorption depth was statistically significantly increased from 25 to 30 μm, and the skin absorption rate was 1.25 to 1.5 μm/min.

The results of improvement of skin absorption of the cosmetic composition of the inventive concept are summarized as shown in Table 5 below.

TABLE 5
Skin Absorption

| | Non-use of test | | | After use of test | |
|---|---|---|---|---|---|
| | product | | | product ± essence | |
| | Average ± standard | After use of essence | | Average ± standard | |
| Depth | deviation | Average ± standard | Comparison | deviation | Comparison |
| (μm) | (μg/cm³) | deviation (μg/cm³) | of groups^{a} | (μg/cm³) | of groups^{a} |
| 0 | −4.313 ± 67.538 | 186.008 ± 103.780 | 0.000+ | 374.976 ± 161.695 | 0.000+ |
| 5 | −2.037 ± 16.502 | 29.372 ± 17.742 | 0.000+ | 96.110 ± 157.359 | 0.000+ |
| 10 | 3.079 ± 7.590 | 9.842 ± 7.599 | 0.007+ | 36.459 ± 77.933 | 0.001+ |
| 15 | 7.057 ± 3.033 | 10.312 ± 2.609 | 0.001+ | 16.194 ± 14.231 | 0.000+ |
| 20 | 7.625 ± 2.382 | 10.519 ± 1.993 | 0.001+ | 12.505 ± 3.150 | 0.000+ |
| 25 | 8.369 ± 1.814 | 10.431 ± 2.496 | 0.004+ | 11.946 ± 3.095 | 0.000+ |
| 30 | 10.978 ± 5.289 | 12.144 ± 3.433 | 0.073 | 12.968 ± 5.839 | 0.025+ |
| Total | 30.757 ± 93.106 | 268.629 ± 120.730 | 0.000+ | 561.157 ± 393.225 | 0.000+ |
| absorption | | | | | |

| Skin absorption depth | 25 | 30 |
|---|---|---|
| (μm) | | |
| Skin absorption rate | 1.25 | 1.50 |
| (μm/min) | | |

Comparison of groups^{a}+: p < 0.05 by Wilcoxon signed ranks test

### Experimental Example 4. Improvement of Skin Roughness

### 4-1. Test Subject Information

The information of the test subjects who participated in the test is as in Table 4 above.

### 4-2. Measurement Method

To measure skin roughness, that is, skin texture, a cheek area of a face was photographed before and after use of the test product using Antera 3D. A specific area of the captured picture was designated and then a degree of roughness was analyzed to be used as evaluation data, and a unit of the analyzed skin roughness was Ra. Because the analysis value and a degree of improvement of skin roughness (skin texture) are inversely proportional to each other, a lower analysis value means that the skin roughness (skin texture) is improved.

### 4-3. Measurement Result

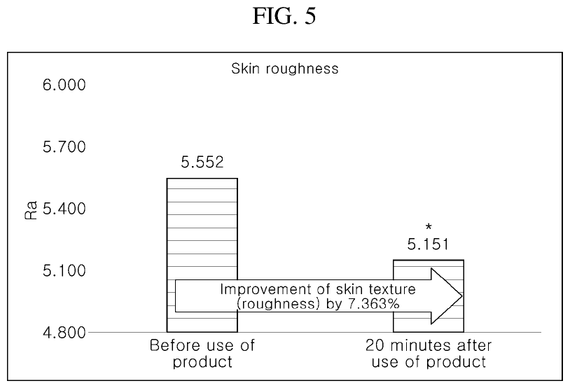

As a result of measuring skin roughness, that is, skin texture, after use of the test product, it was confirmed that the skin roughness analysis value decreased statistically significantly immediately after use of the product as compared with before use of the product (p<0.05) (FIG. 5).

The skin roughness measurement results are shown in Table 6 below.

TABLE 6
Change in skin roughness

| | | | Significance |
|---|---|---|---|
| | Average ± Standard | | probability^{b} |
| Time | deviation (Ra) | Change rate^{a} (%) | (value p) |
| Before use of | 5.552 ± 0.789 | — | — |
| product | | | |
| After use of | 5.151 ± 0.829 | −7.363 | 0.003* |
| product | | | |

$Change {rate}^{a} ( \% ) = {\sum }_{i = 1}^{n} \frac{\begin{matrix}n time after radiation of ultraviolet ray i - \\ before radiation of ultraviolet ray i\end{matrix}}{before radiation of ultraviolet ray i} * 100 / n ,$ n = the number of test subjects
Significance probability\hat{ }^{b} (value p)
‡: p <0.05 by Paired samples T test

Because a lower skin roughness analysis value means that there is an effect of improving skin texture, a change rate marked with a negative value indicates a degree of improvement of skin texture (roughness). This suggests that the cosmetic composition of the inventive concept has an excellent effect of improving skin roughness.

### Experimental Example 5. Evaluation of Compliance

The test subjects were instructed to fill out a compliance log distributed with the test product to determine whether the test product was used when the test product was used according to a usage and a frequency of uses. The subjects who used less than 80% of a compliance of 100% of a total use of the test product were excluded from the measurement data analysis value.

The product use compliance is as shown in Table 7 below.

TABLE 7

| | |
|---|---|
| Average compliance | 99.107% |

### Experimental Example 6. Evaluation of Adverse Reaction

For safety of the test subjects, the occurrence and severity of adverse reactions were checked at each visit.

As a result of confirming the adverse reaction to the product use part at each visit to the test subject, no special adverse skin reaction was reported on the product use part during a test product use period (Table 8).

TABLE 8

| | Adverse skin reaction | | | | | | | |
|---|---|---|---|---|---|---|---|---|
| | | | | | | Burn- | | |
| | | | | | | ing | | |
| | | | | | | sensa- | Stiff- | Ting- |
| Time | Erythema | Edema | Scale | Itch | Sting | tion | ness | ling |
| 7 days | — | — | — | — | — | — | — | — |
| after | | | | | | | | |
| use of | | | | | | | | |
| product | | | | | | | | |
| 14 | — | — | — | — | — | — | — | — |
| days | | | | | | | | |
| of use | | | | | | | | |
| of | | | | | | | | |
| product | | | | | | | | |

Step = 1: weak, 2: middle, 3: severe

### Experimental Example 7. Evaluation of Test Subject Questionnaires

After using the test product, the survey was directly completed on a 5-point scale (5: very satisfied, 4: satisfied, 3: normal, 2: unsatisfied, 1: very unsatisfied) for a degree of preference for the skin improvement effectiveness of the product and a 5-point scale (5: very much, 4: so, 3: average, 2: not so, 1: very not so) for the degree of preference, and the number of test subjects who answered 3 points or more was expressed as a percentage (positive response rate).

As a result, it was confirmed that the degree of skin turnover acceleration and a regeneration of skin improvement was effective at 4 points or more, and the skin absorption amount, absorption depth, and absorption rate were improved, and skin roughness was also improved, and it was also confirmed that a positive response rate was 100% in terms of preference such as feeling of use, fragrance, and purchase intention (Tables 9 and 10).

TABLE 9
Evaluation of test subject questionnaires after use of test product

| | Average ± Standard | Ratio of positive |
|---|---|---|
| Items | deviation | response (%) |
| Degree of skin turnover | 4.150 ± 0.489 | 100.000 |
| promotion effect | | |
| Improvement of skin | 4.100 ± 0.641 | 100.000 |
| regeneration | | |
| Satisfied with feeling of | 4.100 ± 0.788 | 100.000 |
| using product | | |
| Satisfied with smell of | 3.650 ± 0.875 | 95.000 |
| product | | |
| Willing to recommend | 3.850 ± 0.671 | 100.000 |
| product | | |
| Willing to purchase | 4.000 ± 0.562 | 100.000 |
| product | | |
| Satisfied with product | 4.000 ± 0.649 | 100.000 |
| overall | | |

5 point scale of effectiveness evaluation - 5: very satisfied, 4: satisfied, 3: normal, 2: unsatisfied, 1: very unsatisfied
Ratio of positive response (%) - Percentage of the number of test subjects who marked three points or more (normal, satisfied, very satisfied)

TABLE 10
Evaluation of test subject after use of test product

| | Average ± Standard | Ratio of positive |
|---|---|---|
| Items | deviation | response (%) |
| Improvement of skin | 3.950 ± 0.394 | 100.000 |
| absorption amount, | | |
| absorption depth, and | | |
| absorption speed | | |
| Improvement of skin | 4.050 ± 0.510 | 100.000 |
| texture (roughness) | | |
| Satisfied with feeling of | 3.950 ± 0.686 | 100.000 |
| using product | | |
| Satisfied with smell of | 3.850 ± 0.587 | 100.000 |
| product | | |
| Willing to recommend | 4.000 ± 0.562 | 100.000 |
| product | | |
| Willing to purchase | 4.000 ± 0.562 | 100.000 |
| product | | |
| Satisfied with product | 4.000 ± 0.605 | 100.000 |
| overall | | |

5 point scale of effectiveness evaluation - 5: very satisfied, 4: satisfied, 3: normal, 2: unsatisfied, 1: very unsatisfied
Ratio of positive response (%) - Percentage of the number of test subjects who marked three points or more (normal, satisfied, very satisfied)

### Experimental Example 8. Evaluation of Safety

The test product was patched to the skin for 24 hours, and the skin reaction and average skin reactivity (mean score) at the test part 30 minutes, 24 hours, and 48 hours after removal of the patch were evaluated.

The results of the skin patch test on test subjects are shown in Table 11 below.

TABLE 11

| | | | | | Average |
|---|---|---|---|---|---|
| Name of | Number of | Skin reaction | | | skin |
| test material | reactors | after 30 hr. | after 24 hr. | After 48 hr. | reaction |
| Test | 0 | 0.0 | 0.0 | 0.0 | 0.00 |
| product | | | | | |

As a result, no irritation was observed in the test product 30 minutes, 24 hours, and 48 hours after removing the patch. The average skin reactivity was 0.00, which was determined to be non-irritating according to a criterion.

### Experimental Example 9. Analysis of Statistical Results

To determine significance of the skin measurement change value between the test product use part and the test product non-use part, it was verified by using SPSS 23.0 that is a statistical analysis program. Statistical analyses ware performed after excluding the dropout data during the test, and the statistical significance was confirmed when the significance probability was p<0.05 in the 95% reliability section.

For comparison of test product use parts and test product non-use parts, normality test satisfaction was analyzed with the Independent T-test (parametric method), and normality test dissatisfaction was analyzed with the Mann-Whitney U test (non-parametric method).

For comparison before and after using the test product, normality test satisfaction was analyzed with the Paired Samples T-test (parametric method), and normality test dissatisfaction was analyzed with the Wilcoxon signed ranks test (non-parametric method).

Through this, it is suggested that the cosmetic composition containing microneedles impregnated with cica extracts manufactured by the manufacturing method of the inventive concept as active ingredients provides an elasticity and a regeneration to the skin, and a skin turnover, a skin absorption amount, an absorption depth, and an absorption rate are improved, a skin texture, that is, a skin roughness is smoothened, and an excellent compliance and an excellent safety are shown.

An aspect of the inventive concept provides a cosmetic composition including microneedles impregnated with a cica extract as an active ingredient, and a method for manufacturing the same, and the composition provides elasticity and regenerative power to the skin, and has an excellent effect of improving skin turnover, skin absorption amount, absorption depth, absorption rate, and smoothing skin texture, that is, skin roughness whereby the cosmetic composition may be usefully used.

Although the embodiments of the inventive concept have been described above, it may be understood that the inventive concept is not limited to the embodiments and may be manufactured in various different forms, and an ordinary person in the art, to which the inventive concept pertains, the inventive concept may be carried out in other detailed forms without charging technical spirits or essential features of the inventive concept. Therefore, the above-described embodiments should be construed to be exemplary and not limited in all aspects.

What is claimed is:

1. A cosmetic composition comprising microneedles impregnated with cica extract as an active ingredient.

2. The cosmetic composition of claim 1, wherein a content ratio of the cica extract and microneedles is 0.5 to 2:98 to 99.5 parts by weight.

3. The cosmetic composition of claim 1, wherein the microneedles have lengths of 200 to 300 μm and diameters of 10 to 20 μm.

4. The cosmetic composition of claim 1, wherein pHs of the microneedles are 7 to 8.

5. The cosmetic composition of claim 1, wherein the microneedles are originated from sponges.

6. The cosmetic composition of claim 5, wherein the sponges are Spongilla lacustris, Spongilla fragilis leidy or Ephydatia fluviatilis.

7. The cosmetic composition of claim 1, wherein the cosmetic composition improves a turn-over of skin.

8. The cosmetic composition of claim 1, wherein the cosmetic composition expedites improvement of a regeneration of skin.

9. The cosmetic composition of claim 1, wherein the cosmetic composition expedites improvement of a roughness of skin.

10. A method for manufacturing a cosmetic composition, the method comprising:
i) collecting sponge and then hydrolyzing them;
ii) separating microneedles by sieving the hydrolyzed sponges;
iii) drying the microneedles; and
iv) agitating the dried microneedle and cica extract under a reduced pressure to impregnate the cica extract into inner tubes of the microneedles.
